(19) United States
(12) Patent Application Publication
(10) Pub. No.: US 2019/0060115 A1
(43) Pub. Date: Feb. 28, 2019

(54) HANDHELD THERMAL THERAPY DEVICE

(71) Applicant: MG Therapies, Inc., Del Mar, CA (US)

(72) Inventors: Donald Novkov, Encinitas, CA (US); David McMahon, Del Mar, CA (US)

(21) Appl. No.: 16/111,859

(22) Filed: Aug. 24, 2018

Related U.S. Application Data
(60) Provisional application No. 62/550,655, filed on Aug. 27, 2017.

(51) Int. Cl. A61F 9/007; A61F 7/00; A61H 15/00

(52) U.S. Cl. CPC A61F 9/00718; A61F 7/007; A61H 15/0092; A61F 2007/0071; A61F 2007/0093; A61F 2007/0075; A61H 2201/0285; A61F 2007/0078; A61F 2007/0096; A61H 2201/0207; A61H 2201/0214; A61H 2201/0228; A61F 2007/0004

(57) ABSTRACT

This invention discloses a handheld device to provide thermal therapy to tissue by contacting a surface heated with thermal energy to a patient's tissue. Thermal energy is continuously provided during operation. The handheld device comprises an assembly to provide heat operatively connected to a removable thermal energy applicator, the applicator is configured to provide different treatments, including heat application, debridement, and expression of the treated tissue or gland.

### CROSS-REFERENCE TO RELATED APPLICATIONS

This application claims the benefit of U.S. Provisional Application No. 62/550,655, filed Aug. 27, 2017. Each of the above-referenced patent applications is incorporated by reference in its entirety.

### BACKGROUND OF THE INVENTION

### Field of the Invention

This application concerns the use of a handheld device to provide thermal therapy for patients. In particular, this handheld device provides thermal therapy to patient's tissue surface including eye surfaces.

### Description of the Related Technology

Thermal therapy refers to the application of heat or temperature variation to tissues to stimulate physiological activities. Thermal therapy may also be used for tumor ablation and to increase or decrease blood flow to treated tissue by inducing vascular constriction or dilation in the treated area. Tissues receiving thermal therapy may be in various regions across the human or animal body.

Thermal therapy delivery is dependent on the contact surface between the device used for thermal therapy and the patient's tissue. There is a need for a thermal therapy delivery device that is flexible in providing different contact surfaces as well as different contact mechanisms. The delivery of thermal therapy may also be in conjunction with other activities such as massage, expression or debridement of affected tissue.

The ability to deliver consistent treatment sessions is clinically valuable in thermal therapy. Length of treatment, time interval between treatments, temperature, temperature variation, and thermal therapy in combination with other therapies are parameters that may be controlled for effective treatments. Monitoring and tracking of these parameters allow for results to be repeatedly tracked and investigated.

Thermal therapy devices have several challenges. Where fluids are the means for delivering thermal energy, fluid maintenance is cumbersome. Fluid conduits are relatively large and subject to kinking and blockage. Fluid is also prone to leakage and contamination and requires periodic changing. Sanitization of fluid-containing devices is also laborious.

The invention disclosed herein addresses the problems stated herein and other existing problems in the art of thermal therapy.

### SUMMARY

The present invention relates to a medical device for delivering thermal therapy to tissue. More particularly, the present invention relates to a microprocessor or analog circuit-controlled device that is handheld and powered by either an internal battery or a cable that is connected to an external power source. The device is used to deliver several thermal therapies/treatments to a patient's tissue surface including eye surfaces using various interchangeable thermally-conductive metal thermal energy applicator (instruments) that are heated to various temperatures and in some cases enable compression of tissue by the user's mechanical action. When used to treat eyelids, the eyelid margin and more specifically the Meibomian glands may be treated in order to express fluid and contents from the Meibomian glands.

In a specific embodiment, the present invention relates to the device as used for the treatment of Meibomian Gland Dysfunction (MGD), or posterior blepharitis; a common physiological ailment related to the lack of proper flow of Meibomian gland secretions, resulting in a condition known as Dry Eye Disease (DED). There are standard Debridement and Expression hand held tools that are used to treat MGD. However, these instruments are not thermally controlled. The present invention enables a clinician to conduct these treatments using actively heated instruments that apply specific heat to the patient-contacting surfaces only, along with thermal massage of the eyelid surfaces prior to these treatments using a Thermal Massage Instrument. It has been observed that conducting these treatments utilizing these actively-heated instruments on patient-contacting surfaces is much more comfortable for the patient and facilitates the ‘uncapping’ of clogged or non-performing Meibomian glands and the debridement or removal of epithelial cells from the surface of an eyelid which when followed by the heated expression of the Meibomian glands, enhances the expression of Meibomian fluid and reduces symptoms of DED. It is known that the human eyelid cools very rapidly and that simply heating the eyelid using a warm compress does not enable successful expression of the glands as the temperature of the meibum and the eyelid returns to body temperature within a minute of heat removal. Therefore, active heating of the instruments is required for successful Debridement and Expression of the Meibomian Glands. Passively heating instruments (e.g., pre-heating the tools in an oven) is also inadequate, as heat loss results in rapid reduction in temperature and the temperature is unable to be adequately controlled during the procedures. Actively heating allows heat energy to be transferred into the patient-contacting surface constantly during the procedure in order to maintain approximately constant temperature at that surface and thus obtain optimum results.

The present invention allows for user selectable temperature set points. The embodiment shown allows the user to select any of the available different treatments, each with a specific pre-programmed set point temperature that has been clinically determined to be optimum for the specific therapy/treatment. Further embodiments may include more or less discrete set points. Yet further embodiments may allow for a continuously adjustable set point within a given temperature range. The internal control system of the device regulates the heating element in order to control the temperature at the patient side of the Instrument part based on measurement of the conductive metal temperature on a surface close to the heating element. An empirical algorithm based on test data or a theoretical algorithm is used to adjust the control temperature to whatever is required in order to obtain the set point temperature at the patient-contacting surface of the Instrument part.

Currently there is no known handheld device that provides the combination of these therapies/treatments using actively-heated instruments. The present invention fulfills this need by utilizing newly available technology.

In this invention, there is provided a system for providing thermal therapy, the system comprising:

heater power electronics to control heat generation;

microprocessor electronics to control a heating element, user interaction, and device communication;

a heating element positioned for operative connection with a thermal energy applicator;

a removable thermal energy applicator comprised of at least one contact surface configured to communicate thermal energy between the applicator and the patient's tissue, the removable thermal energy applicator is configured to securely connect to the housing; and

a heat flow adapter configured to operatively connect to the removable thermal energy applicator and the heating element at the adapter-applicator interface;

wherein the heater power electronics, the microprocessor electronics, and the heating element are housed in a housing.

There is provided a system as above, wherein the heating element is a resistive heating pad, a thermoelectric cooler, a resistive heating blanket, or a coil of resistance wire.

There is provided a system as above, wherein the removable thermal energy applicator comprises a contact surface oriented orthogonally with respect to the body of the applicator.

There is provided a system as above, wherein the removable thermal energy applicator comprises a debridement tool.

There is provided a system as above, wherein the removable thermal energy applicator comprises two thermal rollers.

There is provided a system as above, wherein one of the thermal rollers is heated and the other is non-heated, and wherein the non-heated roller is made of metal or soft material such as plastic or elastomer.

There is provided a system as above, wherein the non-heated roller is detachable from the remainder of the removable thermal energy applicator.

There is provided a system as above, wherein pressure exerted to tissue being compressed between two rollers is limited by a physical stop or other mechanisms such as adjustable spring to limit the amount of pressure exerted on the tissue being treated in order to reduce or eliminate the risk of causing scarring or other damage to the tissue.

There is provided a system as above, wherein the removable thermal energy applicator comprises two paddles acting as contact surfaces.

There is provided a system as above, wherein both paddles are thermally controlled.

There is provided a system as above, wherein one paddle is heated and the other paddle is non-heated, and wherein the non-heated paddle is made of metal, plastic or elastomer.

There is provided a system as above, wherein the non-heated paddle is detachable from the remainder of the removable thermal energy applicator.

There is provided a system as above, wherein pressure exerted to tissue being compressed between two paddles is limited by a physical stop or other mechanisms such as adjustable spring to limit the amount of pressure exerted on the tissue being treated in order to reduce or eliminate the risk of causing scarring or other damage to the tissue.

There is provided a system as above, wherein the system is configured to operatively communicate with a remote computing device.

There is provided a system as above, wherein the remote computing device is configured to track operating parameters of the device and output usage information for general data collection, use in clinical treatment, or monetarily charge of the users.

There is provided a system as above, wherein the remote computing device is further configured to communicate with a third-party computing device and receive information from the third-party computing device to control the device.

There is provided a system as above, further comprising a battery housing to contain a battery and battery charging and load sharing electronics.

There is provided a system as above, further comprising a thermal cut out device to serve as a fuse.

There is provided a system as above, further comprising a USB connector for power provision and communication with the microprocessor for software loading.

There is provided a system as above, further comprising a graphical display for displaying information relating to the use of the system.

There is provided a system as above, wherein the graphical display is LED, OLED, or LCD display.

There is provided a system as above, wherein the graphical display allows for user interface with the display.

There is provided a system as above, further comprising a temperature sensor operatively connected to measure temperature created by the heating element and configured to communicate temperature measurements to the microprocessor electronics.

### Abbreviations

DED: Dry Eye Disease
MGD: Meibomian Gland Dysfunction
LCD: Liquid Crystal Display
LED: Light Emitting Diode
OLED: Organic Light Emitting Diode
PCBA: Printed Circuit Board Assembly
RTD: Resistance Temperature Detector
TEC: Thermoelectric Cooler
TEM: Thermoelectric Module
USB: Universal Serial Bus
VDC: Volts of Direct Current

### BRIEF DESCRIPTION OF THE DRAWINGS

FIG. 1A shows an isometric view with the Warming Instrument installed.

FIG. 1B shows an embodiment with a graphical Display.

FIG. 2A shows an isometric view with the regular Expression Instrument installed.

FIG. 2B shows an embodiment with a Thumb Wheel compression force adjustment.

FIG. 2C shows an embodiment with the compression force being limited by a hard stop.

FIG. 3A shows an isometric view with the Debridement Instrument installed and the outer parts hidden.

FIG. 3B is a cross-section view showing the battery and taper fit.

FIGS. 4A and 4B show the Thermal Massage Instrument installed.

FIGS. 4C and 4D show the Debridement Instrument installed.

FIGS. 5A and 5B show the regular Expression Instrument installed.

FIGS. 5C and 5D show the Roller Expression Instrument installed.

FIG. 6A shows the Atraumatic Expression Instrument installed.

FIGS. 6B, 6C and 6D show the separable Atraumatic Expression Instrument installed.

FIG. 7A shows the heating element as either a standard resistive heating blanket or a TEC.

FIG. 7B shows the heating element as a resistive heating blanket.

FIG. 7C shows the heating element as a coil of insulated resistance wire.

### DETAILED DESCRIPTION OF CERTAIN INVENTIVE EMBODIMENTS

This present invention is capable of being embodied in various forms. The description below of several embodiments is made with the understanding that the present disclosure is to be considered as an exemplification of the claimed subject matter and is not intended to limit the attached claims to the specific embodiments illustrated. The headings used throughout this disclosure are provided for convenience only and are not to be construed to limit the claims in any way. Embodiments illustrated under any heading may be combined with embodiments illustrated under any other heading.

As used herein, the verb “to comprise” in this description, claims, and other conjugations are used in its non-limiting sense to mean those items following the word are included, but items not specifically mentioned are not excluded.

Reference to an element by the indefinite article “a” or “an” does not exclude the possibility that more than one of the elements are present, unless the context clearly requires that there is one and only one of the elements. The indefinite article “a” or “an” thus usually means “at least one.” Additionally, the words “a” and “an” when used in the present document in concert with the words “comprising” or “containing” denote “one or more”.

The term “thermal energy applicator” or the “Instrument” refers to a removable part that may be operatively connected and removed from the body of the device, the end of which comes into contact with the patient's tissue.

Embodiments of this application relate to a device that provides a heated surface for purposes of medical treatment or physical therapy. The device contains all the electronics, power conditioning, heating element, and a thermal energy applicator or Instrument. It may also contain a battery, either rechargeable or primary, in which case the device will operate with either internal battery power or power from a cord that is connected to an external power source. The operating voltage may be direct current, less than 30 VDC, typically 5-10 VDC.

The heating element may be a heater pad or heater blanket that contains resistive elements, a heater coil consisting of resistance wire, or any similar Joule heating device that converts electricity into heat using resistance. The heating element may also be a Thermoelectric Cooler (TEC), sometimes referred to as a Thermoelectric Module (TEM), otherwise known as a Peltier, or more descriptively a Peltier device assembly consisting of a plurality of alternating n-type and p-type semiconductors connected electrically in series, arranged such that their thermal output due to the Peltier effect is in parallel upon application of electrical current. The polarity applied to the TEC is such that the hot side is towards the Instrument part.

The device may also contain one or more temperature sensors, such as a thermistor, thermocouple or resistance temperature detector (RTD) for the purpose of measuring the heated surface temperature for feedback into the temperature control system and also in some embodiments for displaying to the user in real time. The device may also contain a thermal cutoff that acts as a fuse that is connected to the electronics in such a manner as to cause power to the heating element to be switched off, using a relay or other means, in the event of an electronics failure that causes excessive heating of the surface.

In an embodiment, the device may further comprise a thermal energy applicator or an Instrument. The Instrument may comprise an elongated body with at least one contact surface configured to communicate thermal energy between the applicator and the patient's tissue. The Instrument may be removed or connected to the body of the device, such that thermal energy may be conducted from the heating element to the Instrument. Engagement of the Instrument to the body of the device may be by levers or by other means. A heat flow adapter may be used to operatively connect the Instrument and the heating element to channel and distribute heat flow.

The Instrument may comprise at least one contact surface adapted for various purposes. The contact surface may be a single contact surface and oriented at different angles with respect to the body of the Instrument. The contact surface may be two contact surfaces and comprise other shapes or other additional functions. The contact surface may comprise paddles, debridement tools, thermal rollers, or unheated paddles. The paddles may be removable, or may be made of plastic or elastomer material to reduce trauma inflicted on the tissue at treatment sites.

The Instrument part, which is the patient contact portion of the device, may be removable from the device. This allows the Instrument to be cleaned and sterilized independently. More significantly, this allows for numerous physical shapes and sizes of interchangeable Instrument parts to be used in the same device. Thus, for one particular use, such as Meibomian Gland Dysfunction treatment, the Instrument part may be of a particular size and shape suitable for contacting the eyelid. As another example, the part size and shape may be made suitable for treatment of another eye disorder based on where the area targeted for treatment is located and the geometric features best suited to treat that disorder.

In one embodiment, the device may be operated completely independently with respect to the number of times it is used and the durations of operation. The user simply turns the device on, selects a treatment, continues to select treatments until the therapy session is completed, and then turns the device off.

In a further embodiment, the device may be operated in a controlled manner, in that the number of times it is used and the durations of operation are recorded, internally within the device and/or in a separate computer, this separate computer being either a designated device that works with the handheld device or a non-specific, commercial computer that may exist in proximity to the device or exists on an external server. Communication between the device and external computers may range from a simple commercial method such as USB, Blue Tooth and Ethernet to more complex electronics such as is used in a smart phone. The record collected may enable a second party that owns, leases, or otherwise has a legal contract with the user to charge a fee based on the number of operating sessions, the durations of sessions, the combination of the sessions or duration of sessions or some other parameters.

In a further embodiment, the device is normally in an inactive state when powered on, waiting for an external communication. The user may then request or purchase a therapy session or sessions, this request being made using the communication methods aforementioned or through any other communication method. In this case, the separate computer may act as a host computer. The host computer may then determine if the request or purchase is authorized, and may potentially process the purchase transaction. The host computer may also be commanded to make the authorization by a third party. If authorized, then the host computer may send a communication to the device which allows it to be operated per the request or purchase. Upon completion of the session or sessions, the device may return to the inactive state.

The record of use may also be stored on a computer network, an internet website, or the Cloud. Furthermore, the device may be programmed such that pre-authorization is required via the internet, Cloud, or other electronic communication methods before the device is able to be operated, thus enabling the aforementioned second party to charge a fee in advance for future therapy/treatment sessions.

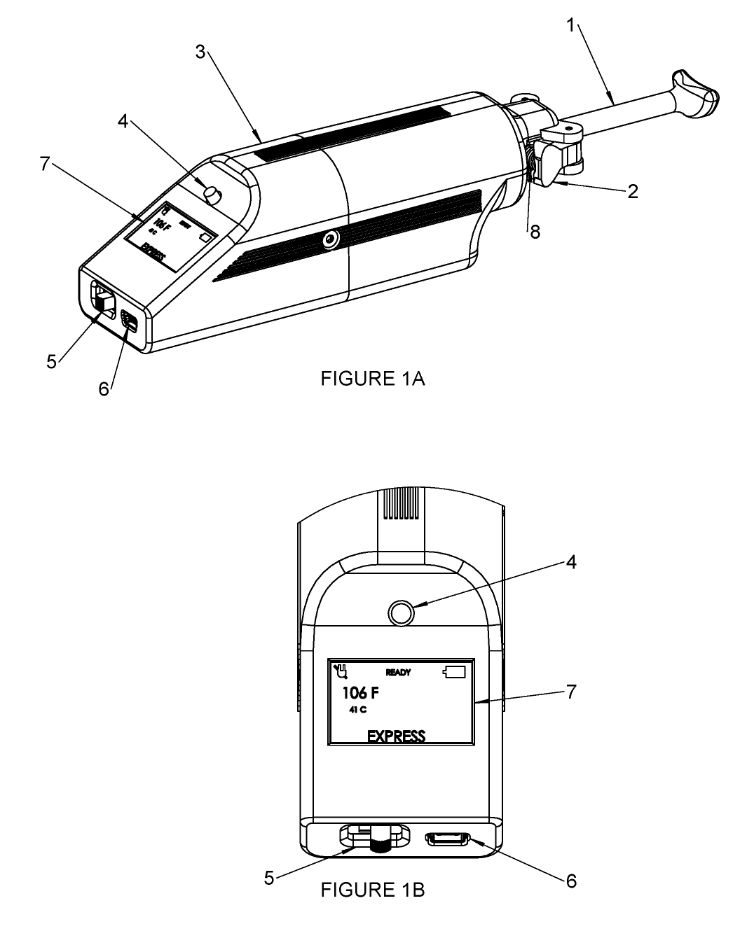

Referring to FIG. 1A, the embodiment of the device as shown comprises of one of the several interchangeable Instruments (thermal energy applicator) (1) that the user installs, the Lever parts (2) that are squeezed to install and released to retain the Instrument, the Housing (3), the Selection button (4) used to select the mode, the Power switch (5) to turn the device on or off, a USB power connector which may also be used to update the software (6), a graphical Display (7), and the Lever springs (8). Apart from the method of using spring-loaded levers, any other suitable means of engaging the parts together may be used. The Instrument (1) shown here is the Thermal Massage Instrument, made of a highly thermally-conductive material, which may have a shape that is designed to transmit heat to relatively large surface areas around the eye to, for example, prepare those areas for subsequent Debridement and Expression treatment. The Thermal Massage Instrument as shown comprises a contact thermal surface oriented orthogonally with respect to the body of the thermal energy applicator. Other angle and/or other shapes of the Thermal Massage Instrument are contemplated.

FIG. 1B shows an embodiment with a graphical Display (7). This Display (7) can be LED, OLED, LCD or any other common type. It may contain a touchscreen to allow user interface with the display. The information it displays can consist of any useful data. In the case shown displayed is battery state, whether the device is plugged into an external power source or not, whether the temperature has stabilized to a steady state (READY), the currently measured temperature in two units (degrees Fahrenheit and degrees Celsius), and what mode the user has selected; these modes being Warming, Debridement or Expression. In some cases, LED indicators may be used instead of a graphical display. Where configured to do so, the Display may also display information on session length, the number of sessions used, or the mode of the sessions, among other information.

Alarms or alerts may be communicated to the user visually via a message on the graphical Display (7) which may blink, or visually via either dedicated discrete LEDs or a blinking of one of the existing LEDs in an embodiment wherein LEDS are used in place of a graphical display as the user interface. In addition, an auditory alarm such as a piezoelectric speaker may be built into the internal electronics to further supplement alarm or alert communication. Additionally, a timer may be built into the graphical display to assist the clinician user in delivering a specific therapy for a specific duration of time.

In the embodiment shown, a USB connector is used to provide power to the device using a USB cord from any standard USB power-providing device. This can be a computer or a simple USB charger. The power cord may be used to charge an internal rechargeable battery, or in another embodiment may be used to power the device without need of an internal battery. Other connectors may be used to connect the device to a power source, either to charge an internal rechargeable battery or to provide power to the device.

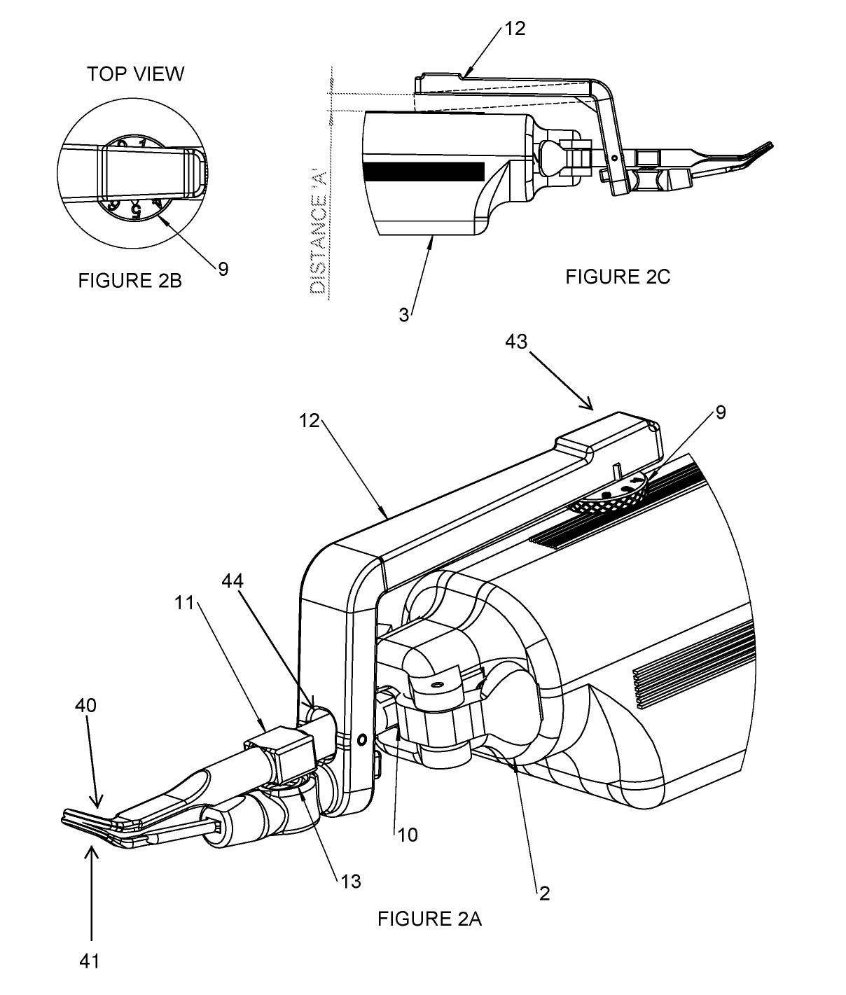

FIG. 2A shows the device where the Instrument is a Thermal Expression Instrument, herein referred to as the Expression Instrument, with the Expression Instrument installed and fully closed. Shown are the thermally controlled part of the Expression Instrument (11) which is made of a highly thermally-conductive material, the indents in that part which engage the levers (10); the unheated part of the Expression Instrument (12) which is made of a combination of a non-thermally-conductive material such as plastic for the main portion including the lever, and either a metallic, plastic or elastomer material for the patient contacting portion (“paddle”) (40); and the Expression Instrument spring (13). The unheated part of the Expression Instrument (12) comprises an aperture (44) wherein the heated part (11) is extended therethrough. The heated and unheated parts contain matched paddles (40), (41) which compress tissue such as an eyelid together when the handle portion (43) of the unheated part is compressed by the user against the spring (13). The heated paddle (40) and the unheated paddle (41) come together upon compression by pressing of the handle (43). The paddles (40), (41) may have small, thin, flat, and smooth surfaces oriented such that they can press together and compress tissue without inflicting injuries to the tissue. The spring (13) loads the Expression Instrument (11) such that it is normally in the open position.

In another embodiment of the Expression Instrument, both paddles may be thermally controlled in order to apply heat equally to the tissue being compressed.

In certain patient cases, including the expression of Meibomian Glands, limiting the compression force imparted to the eyelid is important to reduce trauma during the expression procedure. The amount of force compressing the paddles together may be limited by limiting the stroke of the lever and utilizing the elastic characteristics of the unheated assembly to produce an effective spring force. In one embodiment, limiting of the stroke may be made to be user-adjustable with the addition of a suitable mechanism such as with a thumb screw (9) shown in FIG. 2B, where in this example the distance from the bottom of the thumbscrew to the Housing (3) is adjustable, thus limiting elastic deflection of the unheated assembly (12). In another embodiment, the limit of the stroke may instead be fixed by designing a hard stop in the lever mechanism such as is shown in FIG. 2C whereby the effective spring force is the result of the tip of the lever portion of the unheated assembly (12) deflecting the distance ‘A’ until it contacts the Housing (3), this distance ‘A’ being predetermined to provide adequate maximum paddle compression force in general. Other compression force limiting mechanisms may be utilized instead of utilizing the elastic characteristics of the unheated assembly, for example by using a separate adjustable or non-adjustable compression, extension or torsional spring.

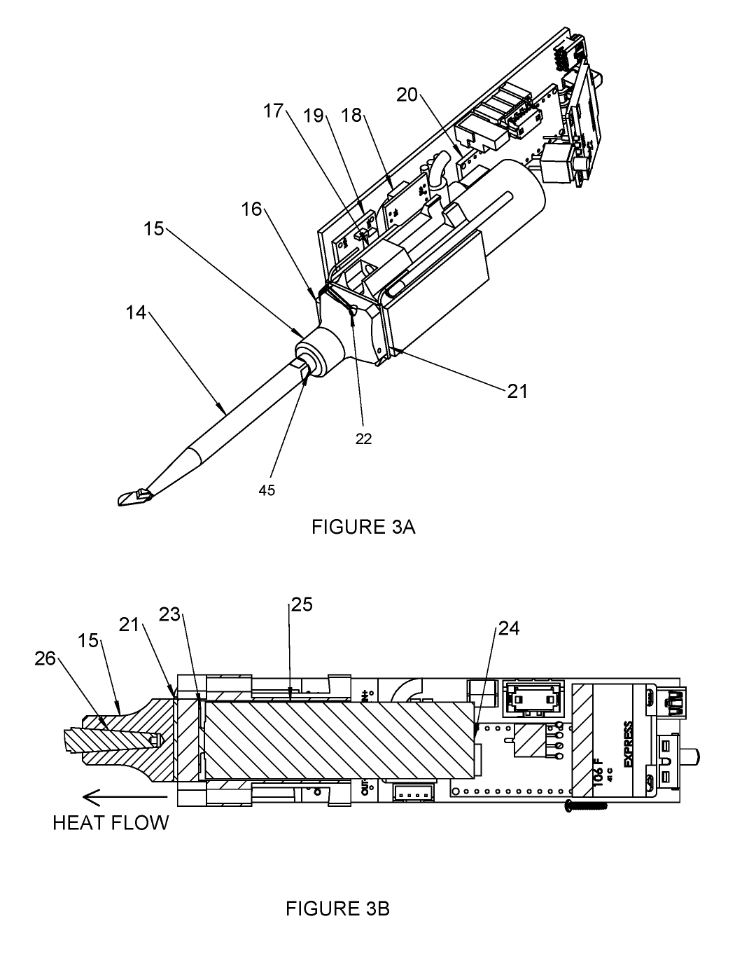

FIG. 3A shows the device and its elements in one embodiment with the outside housing parts hidden. The device is shown with the heated Debridement Instrument (14) installed. This Instrument part, which is made of a highly thermally-conductive material, has a relatively thin and flat shape at the patient-contacting side with sharp edges as necessary for the debridement operation. Other parts shown are a Heat Flow Adapter (15), made of a highly thermally-conductive material, that conducts heat from a heating element to the Debridement Instrument part, a Thermal Cutout device (16) that serves as a fuse in the event of thermal runaway (overheating) of the device, a Battery Housing (17), Battery Charging and Load Sharing electronics (18), Heater Power electronics (19), Microprocessor electronics (20), Heater Pad or TEC (21), and Temperature Sensor (22). If the heating element is a resistive Heater Pad, then the Battery Housing (17) is made of a material that is an electrical insulator, such as plastic. If the heating element is a TEC, then the Battery Housing (17) is made of a thermally-conductive material that is also probably electrically conductive, in which case the battery contained therein requires additional electrical insulation between it and the Battery Housing. The indents (45) for the lever ends, common with all the interchangeable ends with this embodiment of connection type, allow the Debridement Instrument part (14) to be retained in a plurality of rotational directions.

FIG. 3B shows a cross-section view of the aforementioned embodiment with an internal Battery (25). This battery (25) may be primary or rechargeable such as a Lithium-Ion or Nickel-Cadmium type. The direction of heat flow is from the heating element (21) through the Heat Flow Adapter (15) and into the Debridement Instrument part. Thermal interface material or thermal grease may be used to enhance thermal flow between thermally contacting parts. One pole of the Battery (25), in this case the Positive Pole (23) is embedded within the Battery Housing (17). The other pole of the Battery (25), in this case the Negative Pole (24), protrudes outside of the Battery Housing (17), allowing access to the Battery (25) for replacement. The thermal interface is a tapered angle fit (26) of small angle such that there is tight engagement of the two mating surfaces providing good surface contact for efficient heat energy transfer.

In addition to the tapered configuration (26) shown in FIG. 3B, the Instrument to Heat Flow Adapter (15) mating heat transfer surfaces could be a screw thread type engagement, a spring-loaded clamp type or other engagement. Also, the Heat Flow Adapter (15) part could be eliminated by designing the Instrument part with an interface to the heating element that replicates that of the Heat Flow Adapter part, and then by spring force or otherwise pushing the Instrument part and the heating element surfaces together directly. Although the latter could offer better heat transfer efficiency, the fragility and surface pressure requirements of the heating element must be taken into consideration.

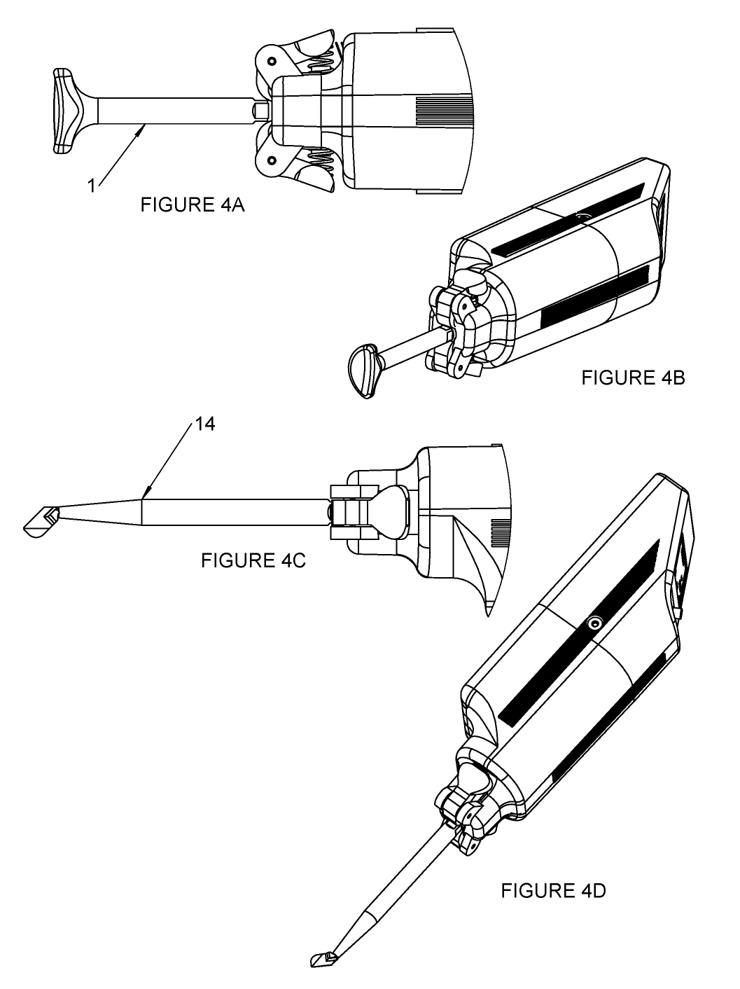

FIG. 4A and FIG. 4B show an embodiment with the Warming Instrument (1) installed. The Warming Instrument (1) in this case has a contact surface oriented primarily or approximately orthogonally to the body of the applicator. FIGS. 4C and 4D show the embodiment with the Debridement Instrument (14) installed.

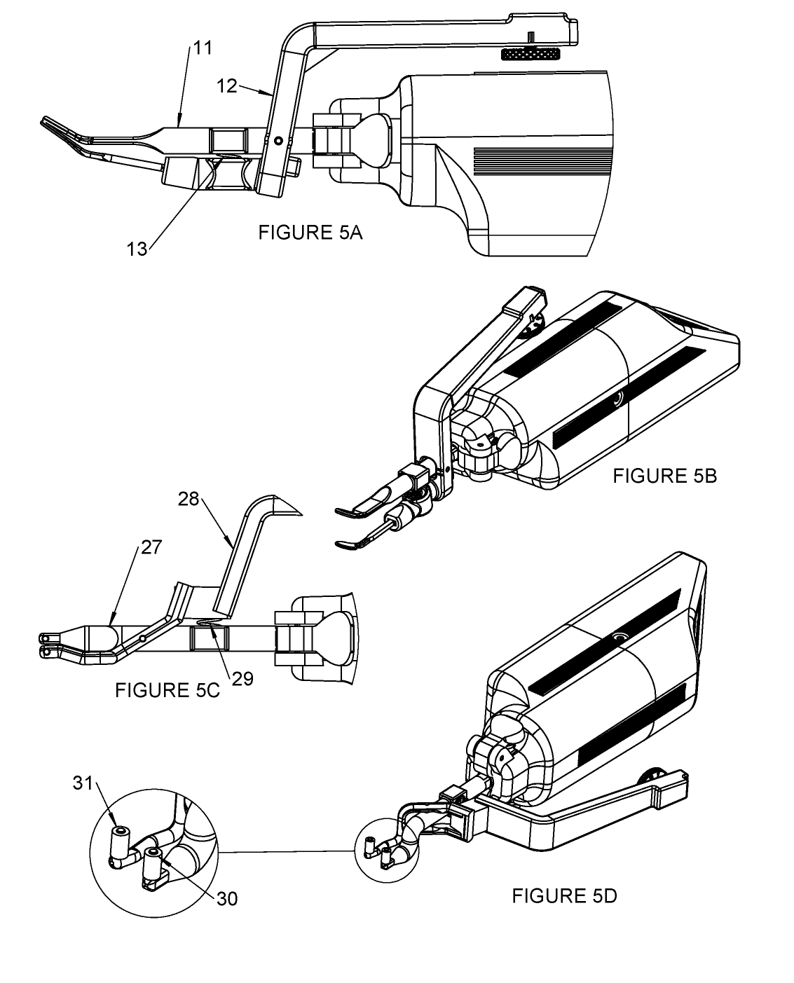

FIGS. 5A and 5B show the embodiment with the regular Expression Instrument (11) installed in the closed and open positions, respectively.

FIG. 5C and 5D show an embodiment with a Roller Expression Instrument installed in the closed and open positions, respectively. This Instrument comprises the Heated Roller Support (27) which is made of a highly thermally-conductive material, the Unheated Roller Support (28), the Spring (29), the Heated Roller (30) which is made of a highly thermally-conductive material and the Unheated Roller (31) which may or may not be made of a thermally-conductive material. The Non-heated Roller (or Unheated Roller) may be made of metal or soft material such as plastic or elastomer. The Heated Roller (30) is stationary while the Unheated Roller (31) moves by pressing of the Unheated Roller Support (28). Optionally, heat may be transferred to both Roller to deliver heat to both sides of the tissue being compressed between the two Heated Rollers. This Roller Expression Instrument operates in the same manner as does the regular Expression Instrument except it has rollers instead of paddles. The use of rollers reduces the friction forces and concentrates the compression force, resulting in a variation of the physical stress patterns of the expression treatment. The Roller Expression Instrument may yield better results than the regular Expression Instrument depending on patient characteristics.

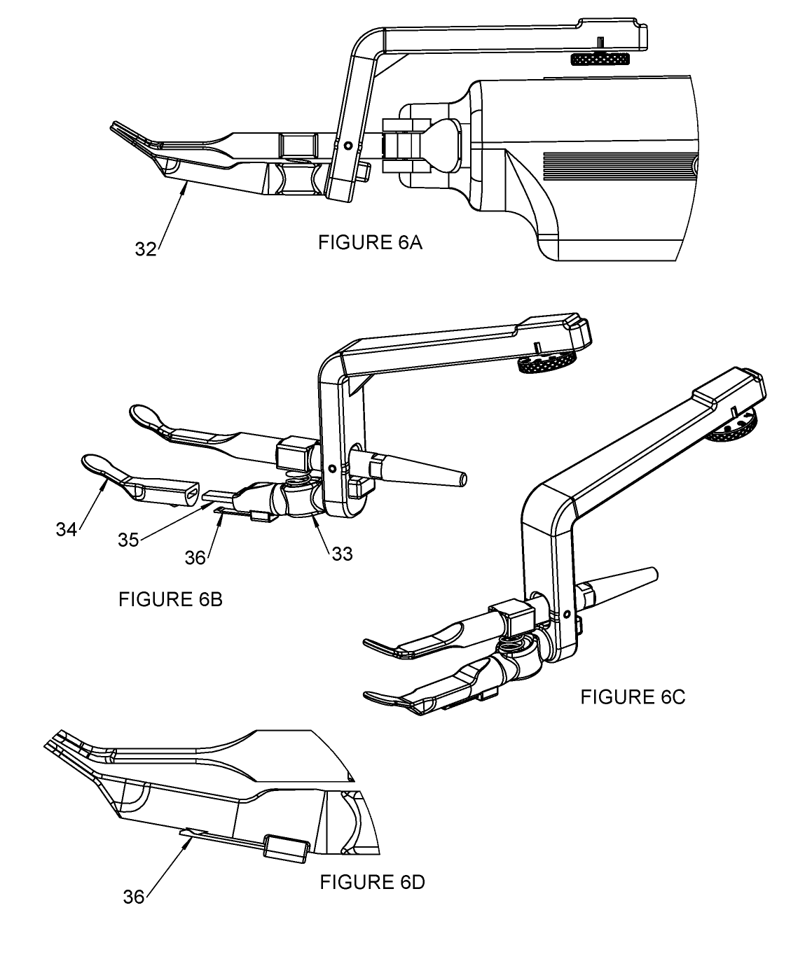

FIGS. 6A through 6D show an embodiment of the Expression Instrument that specifically uses a relatively soft material, such as plastic and or elastomer for the unheated paddle portion for the purpose of providing a softer, or “atraumatic” patient-contacting surface. Alternatively, the unheated paddle (or non-heated) may be made of metal, such as aluminum, among other materials. This Instrument is hereinafter referred to as the Atraumatic Expression Instrument. In an embodiment shown in FIG. 6A, the unheated paddle Instrument portion is integral with the rest of the unheated portion, such that the unheated portion (32) in this case is one single part constructed of the same plastic material or metal such as aluminum, although an elastomer may also be added to the paddle portion on the patient-contacting surface. An Atraumatic Roller Expression Instrument may be constructed in a similar manner.

FIGS. 6B through 6D show an embodiment in which there is a separate detachable unheated paddle part (34), made of plastic, elastomer, metal, or other soft material that is easily installable and removable from the main unheated lever assembly (33). In the example shown, the paddle part (34) mates to the main assembly (33) by virtue of an insert on (35) protruding into a cavity in the paddle part (34), whereby the parts are retained together by a snap-type mechanism (36) as shown in FIG. 6D. Other mechanisms to connect the detachable paddle part (34) to the main unheated lever assembly (33) may be used. The purpose of separate parts is to allow the parts to be constructed of different materials, optimally chosen for their functions. Also, the separate paddle portion may be made sterile and disposable rather than reusable. An Atraumatic Roller Expression Instrument may be constructed in a similar manner.

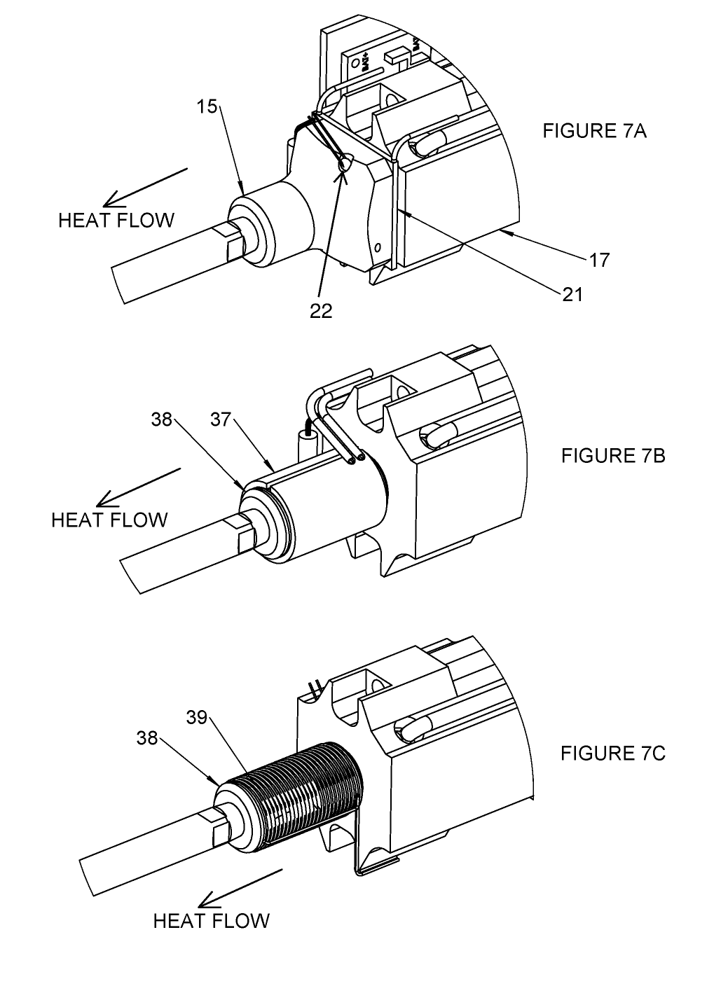

FIG. 7A shows an embodiment with the heating element being either a resistive heating pad or a TEC (21), the material of the Battery Housing (17) and insulation requirements of the Battery being different depending on that choice. Heat is transmitted to the Instrument through the Heat Flow Adapter (15). A Temperature Sensor (22) is located at the Heat Flow Adapter (15) surface.

FIG. 7B shows an embodiment where the heating element is a resistive Heating Blanket (37) wrapped around a differently designed Cylindrical Heat Flow Adapter (38). This Heating Blanket (37) may be made of heating wire or ribbon embedded into a silicone or polyimide substrate and may also contain the temperature sensor such as a thermistor.

FIG. 7C shows an embodiment, which is the preferred heating embodiment, where the heating element is a Coil (39) of resistance wire or ribbon such as Nickel-Chromium or other material designed to produce heat via Joule heating. The wire is either insulated with a thin layer of insulation, or the Heat Flow Adapter is pre-insulated, the bare (uninsulated) windings are spaced accurately apart so as not to short together, and a layer of insulation is applied after winding onto the Heat Flow Adapter. The wire insulation is necessary to prevent electrical shorting but is also thin at the inside surface to facilitate heat flow.

The Temperature Sensor (22) is shown located at the Heat Flow Adapter (15) surface, although in a further embodiment it may be instead located nearer to the patient-contacting surface of the Instrument part if it is desired to control the temperature at a location farther from the heating element and closer to where the heat is conducted to the patient. The benefit of this method is potentially greater temperature accuracy. However, this method presents more difficulty with temperature control due to thermal latency and is either more complicated if the sensor is built into the Instrument part given the electrical connection or is more cumbersome for the user if the sensor would need to be removed/replaced in order to remove/replace the Instrument part.

### Method of Construction

The device is constructed primarily of available off-the-shelf components: microprocessor PCBA (Printed Circuit Board Assembly), Battery charging and load sharing PCBA, Battery, graphical Display, LEDs, switches buttons, various basic PCBA components, connectors, H-bridge controller, inductors, capacitors, resistors, wires, fasteners, etc. Custom parts include several injection-molded plastic parts, the internal machined, cast or 3D-printed metal parts, and the machined or otherwise formed Heated Instrument parts.

The microprocessor operating system is off-the shelf and the firmware application is custom-written if used. If the system is completely analog-based with no microprocessor, then no firmware is required.

The metal parts that conduct heat may be constructed from any metal that has high thermal conductivity. These include several aluminum alloys such as alloys 6101 and 6063 which are relatively inexpensive and light and may be thinly plated with nickel, chrome, or other plating or coating to provide enhanced corrosion resistance. Silver is the best metal for thermal conductivity and may be used to obtain maximum heat transfer at the expense of higher material cost. Pure copper is an excellent material and may likewise be used at the expense of higher machining costs.

Parts may be assembled by hand and/or by automated means. Parts that are connected to each other are done so using any combination of the conventional mechanical fastening techniques (e.g., screws, pins, etc.). PCBAs are constructed per typical commercial manufacturing methods. Operations such as soldering are conventionally performed using standard tools.

Variations and modifications will occur to those of skilled in the art after reviewing this disclosure. The disclosed features may be implemented, in any combination and sub-combination (including multiple dependent combinations and sub-combinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited are hereby incorporated by reference herein in their entireties and made part of this application.

What is claimed is:

1. A system for providing thermal therapy, the system comprising:
heater power electronics to control heat generation;
microprocessor electronics to control a heating element, user interaction, and device communication;
a heating element positioned for operative connection with a thermal energy applicator;
a removable thermal energy applicator comprised of at least one contact surface configured to communicate thermal energy between the applicator and a patient's tissue, the removable thermal energy applicator is configured to securely connect to the housing; and
a heat flow adapter configured to operatively connect to the removable thermal energy applicator and the heating element at the adapter-applicator interface;
wherein the heater power electronics, the microprocessor electronics, and the heating element are housed in a housing.

2. The system of claim 1, wherein the heating element is a resistive heating pad, a thermoelectric cooler used as a heater, a resistive heating blanket, or a coil of resistance wire.

3. The system of claim 1, wherein the removable thermal energy applicator comprises a contact surface oriented primarily orthogonally with respect to the body of the applicator.

4. The system of claim 1, wherein the removable thermal energy applicator comprises a debridement tool.

5. The system of claim 1, wherein the removable thermal energy applicator comprises two thermal rollers.

6. The system of claim 5, wherein one of the thermal rollers is heated and the other is non-heated, and wherein the non-heated roller is made of metal or soft material such as plastic or elastomer.

7. The system of claim 5, wherein the non-heated roller is detachable from the remainder of the removable thermal energy applicator.

8. The system of claim 5, wherein pressure exerted to tissue being compressed between two rollers is limited by a physical stop or other mechanisms such as adjustable spring to limit the amount of pressure exerted on the tissue being treated in order to reduce or eliminate the risk of causing scarring or other damage to the tissue.

9. The system of claim 1, wherein the removable thermal energy applicator comprises two paddles acting as contact surfaces.

10. The system of claim 9, wherein both paddles are thermally controlled.

11. The system of claim 9, wherein one paddle is heated and the other paddle is non-heated, and wherein the non-heated paddle is made of metal, plastic or elastomer.

12. The system of claim 9, wherein the non-heated paddle is detachable from the remainder of the removable thermal energy applicator.

13. The system of claim 9, wherein pressure exerted to tissue being compressed between two paddles is limited by a physical stop or other mechanisms such as adjustable spring to limit the amount of pressure exerted on the tissue being treated in order to reduce or eliminate the risk of causing scarring or other damage to the tissue.

14. The system of claim 1, wherein the system is configured to operatively communicate with a remote computing device.

15. The system of claim 14, wherein the remote computing device is configured to track operating parameters of the device and output usage information for general data collection, use in clinical treatment, or monetarily charge of the users.

16. The system of claim 14, wherein the remote computing device is further configured to communicate with a third-party computing device and receive information from the third-party computing device to control the device.

17. The system of claim 1, further comprising:
a battery housing to contain a battery; and
battery charging and load sharing electronics.

18. The system of claim 1, further comprising a thermal cut out device to serve as a fuse.

19. The system of claim 1, further comprising a USB connector for power provision and communication with the microprocessor for software loading.

20. The system of claim 1, further comprising a graphical display for displaying information relating to the use of the system.

21. The system of claim 20, wherein the graphical display is LED, OLED, or LCD display.

22. The system of claim 20, wherein the graphical display allows for user touchscreen interface with the display.

23. The system of claim 1, further comprising a temperature sensor operatively connected to measure temperature created by the heating element and configured to communicate temperature measurements to the microprocessor electronics.
